(19) United States
(12) Patent Application Publication
(10) Pub. No.: US 2009/0259264 A1
(43) Pub. Date: Oct. 15, 2009

(54) SURGICALLY IMPLANTABLE WIRE CONNECTOR

(75) Inventor: Robert A. Firth, Fraizer Park, CA (US)

Correspondence Address: ALFRED E. MANN FOUNDATION FOR;SCIENTIFIC RESEARCH, PO BOX 905, SANTA CLARITA, CA, 91380, US

(73) Assignee: ALFRED E. MANN FOUNDATION FOR SCIENTIFIC RESEARCH, Santa Clarita, CA (US)

(21) Appl. No.: 12/102,593

(22) Filed: Apr. 14, 2008

(51) Int. Cl. A61N 1/05; H01R 43/04

(52) U.S. Cl. 607/2; 29/861

(57) ABSTRACT

A surgically implantable wire connecting device that is adaptable to accept various diameter wires by selecting pre-wired mandrels, each having a specific candidate diameter wire and length, and attaching the mandrels together in a metal crimp tube. The final assembly during surgery requires a simple crimp of the crimp tube to the adapter tube by using a standard crimp tool. The delicate lead wires that are being attached are not deformed or crushed by the crimp process and are located remote from the crimp.

### BACKGROUND OF THE INVENTION

Crimping a metal connector to retain electrical connection in implantable devices is known in the art of surgically implantable devices, see for example Schulman, et al. U.S. Pat. No. 5,750,926 and Culp U.S. Pat. No. 7,228,624. Crimp connections of small wires have proven difficult to perform during surgery and the small diameter electrical leads are prone to damage or breakage during the crimping procedure.

The instant invention addresses and resolves these problems by a novel and reliable wire attachment device that forms a permanent connection between wire leads and that is implantable surgically for long term exposure to the harsh saline environment of living tissue.

### BRIEF DESCRIPTION OF THE SEVERAL VIEWS OF THE DRAWINGS

FIG. 1 illustrates a top view of the attachment device.

FIG. 2 schematically depicts a cross-sectional view of the attachment device showing the wire leads.

FIG. 3 presents a side view of the attachment device.

FIG. 4 presents an expanded view of the connector device with wire leads shown in the pre-installed position.

FIG. 5 presents a flow diagram for the method of employing the attachment device.

### DETAILED DESCRIPTION OF THE INVENTION

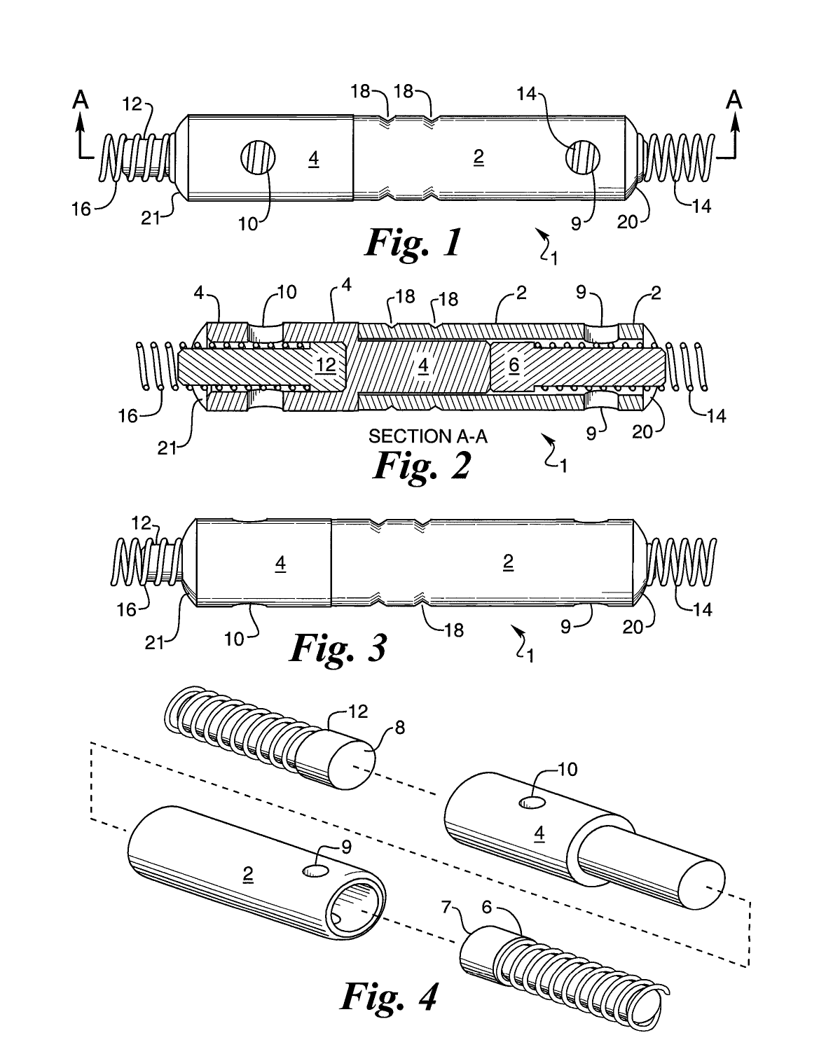

An attachment device 1 is described for attaching wire leads together in a sterile surgical environment having overall dimensions that are less than 20 mm length, excluding the lead wires, and less than 3 mm overall diameter. The device 1 and method of forming an electrical connection are described to connect a first lead 14 insulated electrical wire and a second lead 16 insulated electrical wire, FIG. 1, such as a Peterson or Shimada lead, during implantation surgery, wherein an electrical device, such as a microstimulator or a microsensor, is placed into living tissue, typically muscle or neural tissue, by surgically cutting the skin and underlying tissue.

The leads are very small in diameter and are easily damaged during manipulation and during the formation of a crimp or other connection. The insulated first and the insulated second leads 14, 16 are preferably helically coiled around an electrically conductive mandrel, first lead 14 is coiled around mandrel 6 and second lead 16 is coiled around adapter mandrel 12, FIGS. 2, 3, 4.

Mandrel 6 and adapter mandrel 12 are comprised of a material that is electrically conductive and biocompatible so as to qualify as an implantable device with a long implant life, typically 20 to 80 years, in the hostile warm saline environment of living tissue. While several but not all stainless steels are biocompatible, experimentation has demonstrated that 316 stainless steel is one such preferred candidate.

Mandrel 6 and adapter mandrel 12 have constant diameter rod sections around which the first lead and the second lead, respectively, are helically wound. In a preferred embodiment, the insertion ends 7, 8 of each of mandrel 6 and adapter mandrel 12, respectively, are larger in diameter than the lead winding-containing portion of each mandrel, FIGS. 2, 4. This larger diameter accommodates the helically wound first and second leads to protect the leads during slideable insertion into a receiver tube, FIG. 4.

In a preferred embodiment, prior to surgery the leads 14, 16 are wound around the mandrels 6, 12, respectively. To accommodate the need during surgery to use a longer or shorter lead, a number of leads and mandrel assemblies are prepared in advance of surgery.

Different diameter leads and various type and length leads may be prepared in advance of the final placement by the surgeon, who then can crimp attach the final attachment device 1 in the wet, slippery surgery field with a simple tool.

Mandrel 6, having the prewound first lead 14 helically wrapped around it, is slideably inserted, insertion end 7 first, into crimp tube 2 to a predetermined depth. The depth of insertion is controllable by a number of methods the preferred method being to temporarily insert adapter tube 4 into crimp tube 2 thereby creating a block to control the depth of insertion for mandrel 6 into crimp tube 2. The depth is fixed to allow for a mating of adapter mandrel 12 insertion end 8 with mandrel 6 insertion end 7 when adapter mandrel 12 is fully inserted into crimp tube 2, at which point adapter tube 4 contacts crimp tube 2 along the outer diameter.

Mandrel 6 is fixedly permanently bonded by welding first weld port 9 closed. The resulting weldment holds mandrel 6 in position, but also beneficially melts the insulation from first lead 14 creating electrical communication between first lead 14 and mandrel 6 as well as crimp tube 2. Welding is defined herein as conventional welding, such as laser welding, and also includes laser brazing where a filler material such as type 316 stainless steel is employed.

Adapter mandrel 12 with prewound second lead 16 helically wrapped thereon is slideably inserted, insertion end 8 first, into adapter tube 4. As described previously for first weld port 9, second weld port 10 is similarly welded closed thereby creating a weldment that establishes electrical communication between second lead 16, adapter mandrel 12, and adapter tube 4.

As previously discussed, materials selection is critical to the long-term success of the implanted attachment device 1 as well of each of its components. Selecting the same material for all components of the implantable device eliminates galvanic corrosion as well as the possibility of strain due to thermal expansion mismatch. Type 316 stainless steel is the preferred material for these same reasons.

During surgery the crimp tube 2 and the adapter tube 4 are slideably engaged together by inserting adapter tube 4 cylindrical portion into crimp tube 2 until the adapter tube 4 and crimp tube 2 are in contact at their mating outer diameters. After the surgeon is satisfied that the leads are a satisfactory length and that the assembly 1 is or will be in the desired position in the living tissue, then crimps 18 are formed with a conventional crimping device, thereby permanently engaging the adapter tube 4 in the crimp tube 2. The lead wires are not damaged by crimping, as is the case in known devices, and the electrical connection has already been made between the lead wires 14, 16 and the mandrels, 6, 12, respectively.

A mechanically compliant seal 20 is placed at the interface between crimp tube 2 and mandrel 6, as well as at the interface between adapter tube 4 and adapter mandrel 12 as shown in FIGS. 1, 2, 3. The mechanically compliant seal is comprised of a silicone having a minimum elasticity of 50 durometers on the Shore A scale, thereby preventing migration of saline body fluids into the interface between the two components in which first lead 14 is helically wound. However, the seal 20 also provides strain relief to the first lead 14, thereby assuring that the lead 14 will not fail due to movement at the interface. The mechanically compliant seal 20 is preferably applied pre-surgery, thus eliminating this step during the actual surgical procedure.

A second mechanically compliant seal 21 is placed between the adapter tube 4 and the adapter mandrel 12 for strain relief to the second lead 16 which passes through said second mechanically compliant seal.

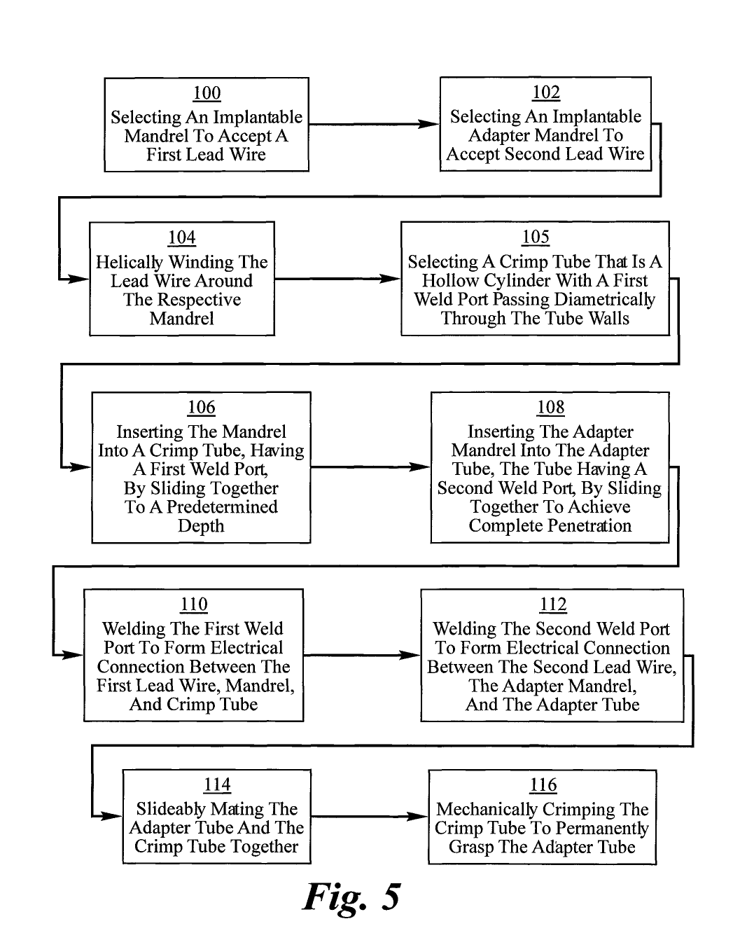

A flow diagram is presented in FIG. 5 describing the method of establishing electrical connectivity between wires 14, 16 during surgery by first selecting an implantable mandrel 6 to accept a first lead wire 14 [step 100]. Then an implantable adapter mandrel 12 is selected to accept second lead wire 16 [step 102]. The first lead 14 and the second lead wire 16 are helically wound around their respective receiving mandrel 6 and adapter mandrel 12 [step 104].

A crimp tube 2 that is a hollow cylinder with a first weld port 9 passing perpendicular to the longitudinal axis of the tube 2 through the walls is selected [step 105]. In a preferred embodiment the crimp tube 2, adapter tube 4 and mandrel 6 are comprised of a stainless steel, preferably type 316 stainless, due to its superior weldability and biocompatibility as well as electrical conductivity and ability to be crimp bonded.

The mandrel 6 with helically wound first lead 14 are slideably inserted into the crimp tube 2, which has a first weld port 9 defined by its cylindrical wall [step 106]. The mandrel 6 is inserted to a predetermined depth that is preferably controlled by previously inserting on a temporary basis adapter mandrel 4, thereby defining the maximum insertion depth for mandrel 6. After mandrel 6 is inserted it is welded into place at weld port 9 by closing weld port 9 by welding [step 110], the adapter mandrel 4 is removed from its temporary position.

The adapter mandrel 12 with helically wound second lead 16 is slideably inserted into adapter tube 4 and welded into place by filling second weld port 10 with weldment material [step 112], thereby establishing electrical contact between lead 16, adapter mandrel 12 and adapter tube 4 [step 108].

The adapter mandrel 4 is slideably inserted into crimp tube 2 to form a mated assembly [step 114]. Finally the complete attachment device 1 is formed by mechanically crimping the crimp tube 2 to permanently grasp the adapter tube with crimp indentations 18 [step 116].

In a preferred embodiment the selecting steps 100, 102, 105, and 106 are selecting a device component that is comprised of stainless steel, in a preferred embodiment type 316 stainless steel is selected.

In a further preferred embodiment the step 106 inserting the mandrel 6 into the crimp tube 2 further involves selecting and applying an mechanically compliant seal 20 to the crimp tube 2 and mandrel 6 at their interface to hermetically seal and to provide strain relief to the first lead 14 which passes through said mechanically compliant seal 20, FIG. 1.

### Glossary

Terms are to be interpreted within the context of the specification and claims. The following terms of art are defined and shall be interpreted by these definitions. Medical terms that are not defined here shall be defined according to The American Heritage Stedman's Medical Dictionary, Houghton Mifflin, 1995, which is included by reference in its entirety. Terms that are not defined here shall be defined according to definitions from the ASM Metals Reference Book, 3^{rd} Edition, 1993, which is included by reference in its entirety.

Biocompatible. The ability of a long-term implantable medical device to perform its intended function, with the desired degree of incorporation in the host, without eliciting any undesirable local or systemic effects in that host. Regulatory agencies require that implanted objects or devices within the human body be biocompatible.

Body. The entire material or physical structure of an organism, especially of a human.

Bond. In welding, brazing, or soldering, the junction of joined parts. Where filler metal is used, it is the junction of the fused metal and the heat-affected base metal.

Butt joint. A joint between two abutting members lying approximately in the same plane.

Cavity. The hollow area within the body, such as a sinus cavity, vagina, mouth, rectum, or ear.

Filler metal. Metal added in making a brazed, soldered, or welded joint.

Foil. Metal in sheet form less than 0.15 mm (0.006 inches) thick.

Hermetic. Completely sealed by fusion, soldering, brazing, etc., especially against the escape or entry of air or gas.

Implant. To embed an object or a device in a body surgically along a surgically created implantation path.

Insert. To place an object or a device into a body cavity.

Interlayer. See Foil.

Joined. Fastened together by brazing, welding, or soldering.

Liquidus. In a phase diagram, the locus of points representing the temperatures at which the various compositions in the system begin to freeze on cooling or finish melting on heating.

Microstimulator. An implantable, biocompatible device having dimensions that are less than about 6 mm diameter and 60 mm in length that is capable of stimulating by electrical signal as well as of sensing electrical signals within living tissue.

Noble metal. A metal with marked resistance to chemical reaction, particularly to oxidation and to solution by inorganic acids.

Roll bonding. The same as roll welding and forge welding. A solid-state process where metals are forced together while hot by applying very high pressure that is asserted by rolls to form plate, sheet or foil material and not complex shapes. No filler material is used to achieve roll bonding.

Soldering. A group of processes that join metals by heating them to a suitable temperature below the solidus of the base metals and applying a filler metal having a liquidus not exceeding 450° C. (840° F.). Molten filler metal is distributed between the closely fitted surfaces of the joint by capillary action.

Solid-state welding. A group of processes that join metals at temperatures essentially below the melting points of the base materials, without the addition of a brazing or soldering filler metal. Pressure may or may not be applied to the joint.

Solidus. In a phase diagram, the locus of points representing the temperatures at which various compositions stop freezing upon cooling or begin to melt upon heating.

Subcutaneous. Located, found, or placed just beneath the skin.

Surgery. A procedure involving the cutting or intrusive penetration of body tissue by cutting or penetration and not by inserting an object or a device into a naturally existing body cavity.

Surgical. Of, relating to, or characteristic of surgeons or surgery.

The invention claimed is:

1. A surgically implantable attachment device for establishing electrical communication of insulated electrically conductive wires, comprising:
a mandrel around which a first lead is helically wound;
said mandrel slideably engaged into a crimp tube that defines a first weld port plug to facilitate weldably attaching said mandrel inside said crimp tube;
said first lead is in electrical communication to said mandrel by welding said first plug weld port closed;
an adapter mandrel around which a second lead is helically wound;
said adapter mandrel slideably engaged inserted inside said adapter tube defining a second weld port plug which facilitates weldably attaching said adapter mandrel by welding said second weld port plug closed;
said second lead in electrical communication with said adapter mandrel by welding said second plug weld port; and
said adapter tube slideably inserted into said crimp tube and engaged therein by crimp deformations of said crimp tube.

2. The surgically implantable attachment device according to claim 1 wherein said device is less than 20 mm in length and less than 3 mm in maximum diameter to facilitate surgical implantation in living tissue.

3. The surgically implantable attachment device according to claim 1 wherein said device is comprised of type 316 stainless steel.

4. The surgically implantable attachment device according to claim 1 wherein said mandrel having said first lead that is helically wound around the mandrel is hermetically sealed to said crimp tube by a mechanically compliant seal for strain relief.

5. A method of connecting electrically conductive wires during surgery comprising the steps of:
selecting a mandrel around which a first lead is helically wound;
selecting an adapter mandrel around which a second lead is helically wound,
slideably inserting said mandrel into a crimp tube that is configured to slideably mate with an adapter tube, said crimp tube wall defining a first weld port;
slideably inserting said adapter mandrel into said adapter tube, said adapter tube wall defining a second weld port;
weldably bonding at the first weld port to establish electrical communication with said first lead, said mandrel and said crimp tube,
weldably bonding at the second weld port to establish electrical communication with said second lead, said adapter mandrel and said adapter tube,
slideably mating said adapter tube with said crimp tube; and
crimping said crimp tube to retain said adapter tube therein.

6. The method according to claim 5 further comprising selecting and applying a mechanically compliant seal to the crimp tube and mandrel for strain relief to the first lead which passes through said mechanically compliant seal.

7. The method according to claim 5 further comprising selecting and applying a second mechanically compliant seal to the adapter tube and adapter mandrel for strain relief to the second lead which passes through said second mechanically compliant seal.

8. The method according to claim 5 further comprising selecting type 316 stainless steel for the mandrel, adapter mandrel, and crimp tube.
